(19) United States
(12) Patent Application Publication
(10) Pub. No.: US 2024/0124825 A1
(43) Pub. Date: Apr. 18, 2024

(54) IMMOBILIZED ENZYMOLYSIS EQUIPMENT FOR RECONSTITUTED RICE MILK POWDER

(71) Applicant: Harbin University Of Commerce, Harbin (CN)

(72) Inventors: Na ZHANG, Harbin (CN); Yang YANG, Harbin (CN); Xin BIAN, Harbin (CN); Chunmin MA, Harbin (CN); Xiaofei LIU, Harbin (CN); Bing WANG, Harbin (CN); Yinyuan HE, Harbin (CN)

(73) Assignee: Harbin University Of Commerce, Harbin (CN)

(21) Appl. No.: 18/357,455

(22) Filed: Jul. 24, 2023

(30) Foreign Application Priority Data
Oct. 13, 2022 (CN) 202211255778.8

(51) Int. Cl. C12M 1/42; C12M 1/02; C12M 3/00

(52) U.S. Cl. CPC C12M 35/04; C12M 23/48; C12M 41/18

(57) ABSTRACT

An immobilized enzymolysis equipment for reconstituted rice milk powder includes a bottom box, a heating device is arranged on an upper surface of the bottom box, and a splash-proof device is arranged on an upper surface of the heating device. Support legs are fixedly connected to corners of a lower surface of the bottom box. A rotating motor is arranged on a bottom surface of an inner cavity of the bottom box. The rotating motor is provided to achieve the effect of rotating a centrifugal device. The centrifugal device is rotationally connected to one side, extending to the upper surface of the bottom box, of the rotating motor, so as to achieve water-milk separation of reconstituted milk powder.

### CROSS-REFERENCE TO RELATED APPLICATIONS

This application claims priority to Chinese Patent Application No. 202211255778.8, filed on Oct. 13, 2022, the content of which is incorporated herein by reference in its entirety.

### TECHNICAL FIELD

The present disclosure relates to the technical field of enzymolysis equipment, and in particular to immobilized enzymolysis equipment for preparing rice milk powder, relating to the technical field of enzymolysis equipment.

### BACKGROUND

Enzymatic hydrolysis refers to the process that organism decomposes large pieces of food with complex molecular structure into absorbable small molecular substances with simple molecular structure through the movement of digestive tract and the enzymolysis of secretions of the digestive glands. For example, nerves and hormones can reinforce each other when acting on the same target cell at the same time. Enzyme is a type of protein produced by organisms and having high efficiency and specific catalytic function. In organisms, the enzyme is involved in catalyzing almost all substance transformation processes, which are closely related to life activities. In vitro, the enzyme can be used as a catalyst for industrial production. However, as most enzymes belong to proteins, and their advanced structures are particularly sensitive to the environment, various factors, such as physical factors, chemical factors and biological factors, may lead to the loss of biological activity. Even under the optimum conditions, the enzyme is about to be gradually inactivated, and the reaction speed gradually decreases as the reaction time increases. Further, the enzyme after reaction cannot be recovered, such that the catalyst can only be produced by a batch method, which is costly for modern biocatalysis industry.

It is necessary to separate water from milk before enzymolysis, and the enzymolysis effect can be produced after the separation of water. However, the enzymolysis equipment on the market cannot achieve the water-milk separation of reconstituted milk powder.

### SUMMARY

In order to achieve the objective above, the present disclosure is implemented through the following technical solutions: Immobilized enzymolysis equipment for reconstituted rice milk powder includes a bottom box. A heating device is arranged on an upper surface of the bottom box, a splash-proof device is arranged on an upper surface of the heating device, and support legs are fixedly connected to corners of a lower surface of the bottom box. A rotating motor is arranged on a bottom surface of an inner cavity of the bottom box. The rotating motor is provided to achieve the effect of rotating a centrifugal device. During the operation of the immobilized enzymolysis equipment, after a test tube is placed on the centrifugal device by an operator, the rotating motor is connected to a power supply and a switch of the rotating motor is turned on, then the rotating motor starts to rotate. The rotating motor penetrates through the bottom surface of the inner cavity of the bottom box and extends to the upper surface of the bottom box. The centrifugal device is rotationally connected to one side, extending to the upper surface of the bottom box, of the rotating motor. The centrifugal device includes a rotating column. The rotating column is rotationally connected to an upper surface of the rotating motor, and is connected to an output end of the rotating motor. A fixed column is fixedly connected to an upper surface of the rotating column. A placement plate is fixedly connected to an upper surface of the fixed column. A limit column is fixedly connected to an upper surface of the placement plate, and an upper placement hole is formed in the upper surface of the placement plate. Through the arrangement of the placement plate and the upper placement hole, during the operation of the immobilized enzymolysis equipment, a test tube filled with the reconstituted milk powder is placed at the upper placement hole formed in the upper surface of the placement plate, such that the test tube can be placed in the centrifugal device, and a centrifugal effect can be achieved under the action of the rotating motor, so as to achieve the enzymolysis effect on the reconstituted milk powder.

Preferably, a fan-shaped support plate is fixedly connected to an outer surface of the fixed column, and a bow-shaped elastic sheet is fixedly connected to one side, away from the fixed column, of the fan-shaped support plate. The bow-shaped elastic sheet is provided to play a role in buffering during the rotation of the test tube. Vibration may be generated during the rotation of the rotating column, and the providing of the bow-shaped elastic sheet may play a certain cushioning effect. A test tube clamping device is arranged at one end, away from the fan-shaped support plate, of the bow-shaped elastic sheet. The test tube clamping device includes a bottom plate of the clamping device. The bottom plate of the clamping device is fixedly connected to one end, away from the fan-shaped support plate, of the bow-shaped elastic sheet. A clamping support box is fixedly connected to an upper surface of the bottom plate of the clamping device, and a bottom placement hole is formed in an upper surface of the clamping support box. A support plate is provided between an inner cavity of the clamping support box and an opposite face of the bottom plate of the clamping device. A fitting box is fixedly connected to an outer surface of the support plate. An adhesive strip is arranged at an inner wall of the fitting box. An arc-shaped elastic sheet is fixedly connected to the outer surface of the support plate, an extrusion column is fixedly connected to one side, close to the fitting box, of the arc-shaped elastic sheet, and the extrusion column is in extrusion fit with the fitting box. The test tube clamping device is provided to clamp and fix the bottom of the test tube, so as to prevent the test tube from flying off the centrifugal device during rotation. During the operation of the immobilized enzymolysis equipment, the test tube filled with the reconstituted milk powder is placed into the centrifugal device from the upper placement hole formed in the upper surface of the placement plate by the operator, and then the test tube is pressed to be clamped into the bottom placement hole formed in the upper surface of the clamping support box. The bottom of the test tube extrudes the arc-shaped elastic sheet, and the extrusion column and the fitting box extrude each other during the extrusion. Due to the adhesive strip on the inner wall of the fitting box, the adhesive strip and the extrusion column extrude each other, such that the extrusion column is firmly extruded at an inner cavity of the fitting box, making the test tube fixed firmly.

Preferably, the heating device includes a base of the heating device. The base of the heating device is fixedly connected to the upper surface of the bottom box. A heating motor is arranged on an upper surface of the base of the heating device. A conductive plate is connected to a side surface of the heating motor. One end, away from the heating motor, of the conductive plate is provided with a heating plate, and the heating plate is connected to an output end of the heating motor. The heating motor and the heating plate are provided to keep the test tube at a certain temperature. As it is necessary to keep a certain temperature during the growth of enzyme bacteria in the reconstituted rice milk powder, the heating motor and the heating plate are provided to control the temperature around the test tube. During the operation of the immobilized enzymolysis equipment, the heating motor is connected to a power supply by an operator, and then a switch on the heating motor is turned on. The heating motor starts to work, the heating plate dissipates heat to keep the test tube at a certain temperature. A limit top plate is fixedly connected to an upper surface of the heating motor, and an anti-collision strip is arranged on an outer surface of the limit top plate. A partition plate is fixedly connected to one side, away from the anti-collision strip, of the outer surface of the limit top plate, and the other end of the partition plate is fixedly connected to a side surface of the base of the heating device. A fan-shaped limit plate is fixedly connected to the upper surface of the base of the heating device. The anti-collision strip is provided to prevent the centrifugal device from colliding with the limit top plate during rotation, because the collision with the limit top plate may lead to nonuniform centrifuging of materials in the test tube, the arrangement of the anti-collision strip can keep the centrifugal device stable when colliding with the limit top plate. The fan-shaped limit plate is arranged around the fixed column, so as to play a role in limiting the centrifugal device.

Preferably, the splash-proof device includes a bottom shell of the splash-proof device. The bottom shell of the splash-proof device is fixedly connected to the outer surface of the limit top plate, a top cover of the splash-proof device is sleeved at an inner cavity of the bottom shell of the splash-proof device, and a top limit box is fixedly connected to a lower surface of the top cover of the splash-proof device. The top limit box is in frictional fit with the limit column. A grip is fixedly connected to an upper surface of the top cover of the splash-proof device. A clamping groove is formed in an upper surface of the bottom shell of the splash-proof device, and a clamping column is fixedly connected to an outer surface of the top cover of the splash-proof device. The clamping column is in extrusion fit with the clamping groove formed in the upper surface of the bottom shell of the splash-proof device. A limit block is fixedly connected to a lower surface of the clamping column, and a limit soft sheet is arranged at a side surface of the limit block. The limit soft sheet is in extrusion fit with the clamping groove formed in the upper surface of the bottom shell of the splash-proof device. The splash-proof device is provided to prevent liquid in the test tube from splashing everywhere during the rotation of the centrifugal device. During the operation of the immobilized enzymolysis equipment, after the test tube is placed on the centrifugal device, the top cover of the splash-proof device is placed on the upper surface of the bottom shell of the splash-proof device by the operator. The limit block and the limit soft sheet are placed at the clamping groove formed in the upper surface of the bottom shell of the splash-proof device, and the limit block can be tightly clamped to the clamping groove formed in the upper surface of the bottom shell of the splash-proof device through the limit soft sheet, thus achieving a splash-proof effect.

The present disclosure provides immobilized enzymolysis equipment for reconstituted rice milk powder. The present disclosure has the following beneficial effects:

1. According to the immobilized enzymolysis equipment for reconstituted rice milk powder, the rotating motor is provided to achieve an effect of rotating the centrifugal device. During the operation of the immobilized enzymolysis equipment, after a test tube is placed on the centrifugal device by an operator, the rotating motor is connected to a power supply and a switch of the rotating motor is turned on, then the rotating motor starts to rotate. Through the arrangement of the placement plate and the upper placement hole, during the operation of the immobilized enzymolysis equipment, the test tube filled with the reconstituted milk powder is placed at the upper placement hole formed in the upper surface of the placement plate by the operator, such that the test tube can be placed in the centrifugal device, and a centrifugal effect can be achieved under the action of the rotating motor, so as to achieve the enzymolysis effect on the reconstituted milk powder.

2. According to the immobilized enzymolysis equipment for reconstituted rice milk powder, the bow-shaped elastic sheet is provided to play a role in buffering during the rotation of the test tube. Vibration may be generated during the rotation of the rotating column, and the providing of the bow-shaped elastic sheet may play a certain cushioning effect. The test tube clamping device is provided to clamp and fix the bottom of the test tube, so as to prevent the test tube from flying off the centrifugal device during rotation. During the operation of the immobilized enzymolysis equipment, the test tube filled with the reconstituted milk powder is placed into the centrifugal device from the upper placement hole formed in the upper surface of the placement plate by the operator, and then the test tube is pressed to be clamped into the bottom placement hole formed in the upper surface of the clamping support box. The bottom of the test tube extrudes the arc-shaped elastic sheet, and the extrusion column and the fitting box extrude each other during the extrusion. Due to the adhesive strip on the inner wall of the fitting box, the adhesive strip and the extrusion column extrude each other, such that the extrusion column is firmly extruded at an inner cavity of the fitting box, making the test tube fixed firmly.

3. According to the immobilized enzymolysis equipment for reconstituted rice milk powder, the heating motor and the heating plate are provided to keep the test tube at a certain temperature. As it is necessary to keep a certain temperature during the growth of enzyme bacteria in the reconstituted rice milk powder, the heating motor and the heating plate are provided to control the temperature around the test tube. During the operation of the immobilized enzymolysis equipment, the heating motor is connected to a power supply by an operator, and then a switch on the heating motor is turned on. The heating motor starts to work, the heating plate dissipates heat to keep the test tube at a certain temperature. The anti-collision strip is provided to prevent the centrifugal device from colliding with the limit top plate during rotation, because the collision with the limit top plate may lead to nonuniform centrifuging of materials in the test tube, the arrangement of the anti-collision strip can keep the centrifugal device stable when colliding with the limit top plate. The fan-shaped limit plate is arranged around the fixed column, so as to play a role in limiting the centrifugal device.

4. According to the immobilized enzymolysis equipment for reconstituted rice milk powder, the splash-proof device is provided to prevent liquid in the test tube from splashing everywhere during the rotation of the centrifugal device. During the operation of the immobilized enzymolysis equipment, after the test tube is placed on the centrifugal device, the top cover of the splash-proof device is placed on the upper surface of the bottom shell of the splash-proof device by the operator. The limit block and the limit soft sheet are placed at the clamping groove formed in the upper surface of the bottom shell of the splash-proof device, and the limit block can be tightly clamped to the clamping groove formed in the upper surface of the bottom shell of the splash-proof device through the limit soft sheet, thus achieving a splash-proof effect.

### BRIEF DESCRIPTION OF THE DRAWINGS

FIG. 1 is a schematic diagram of an overall structure in accordance with the present disclosure.

FIG. 2 is a schematic diagram of a sectional structure in accordance with the present disclosure.

FIG. 3 is a schematic diagram of a structure of a centrifugal device in accordance with the present disclosure.

FIG. 4 is a schematic diagram of a sectional structure of a centrifugal device in accordance with the present disclosure.

FIG. 5 is a schematic diagram of a structure of a test tube clamping device in accordance with the present disclosure.

FIG. 6 is a schematic diagram of a detailed structure of a test tube clamping device in accordance with the present disclosure.

FIG. 7 is a schematic diagram of a structure of a heating device in accordance with the present disclosure.

FIG. 8 is a schematic diagram of a structure of a splash-proof device in accordance with the present disclosure.

### DETAILED DESCRIPTION OF THE EMBODIMENTS

The present disclosure is further described below with reference to the accompanying drawings and embodiments. Embodiments of the present disclosure are given for the purpose of illustration and description and are not exhaustive or limited to the disclosed form. Many modifications and changes are apparent to those of ordinary skill in the art. Embodiments have been selected and described to illustrate the principles and practical applications of the present disclosure better, and to enable those of ordinary skill in the art to understand the present disclosure and thus design various embodiments with various modifications suitable for a particular use.

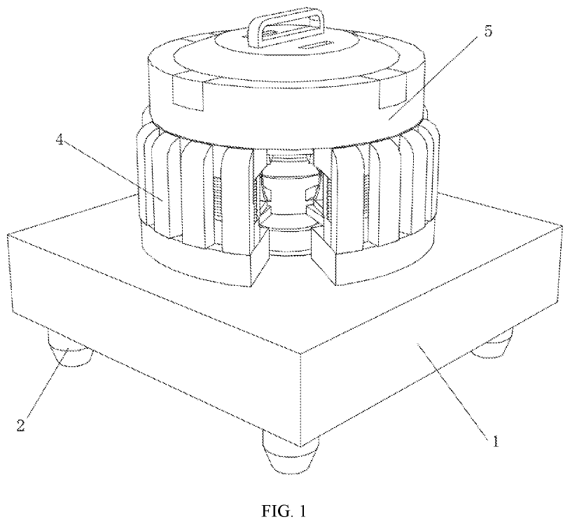

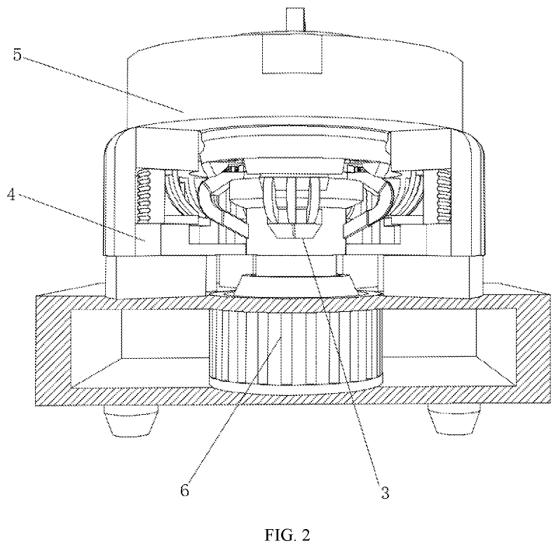

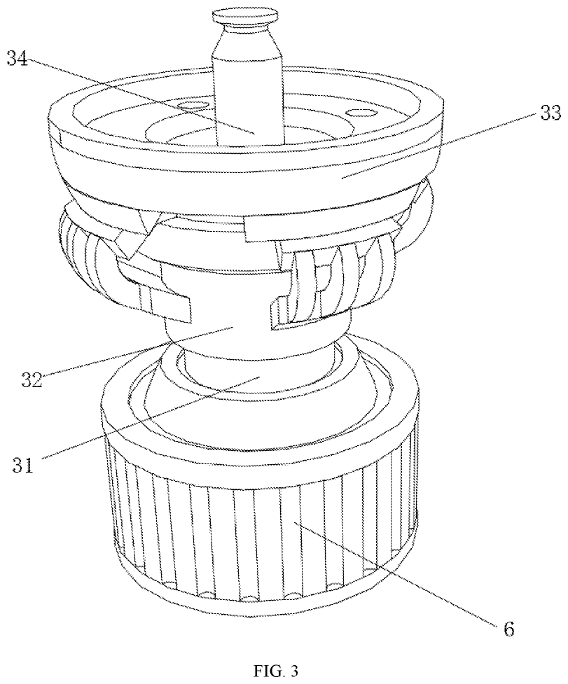

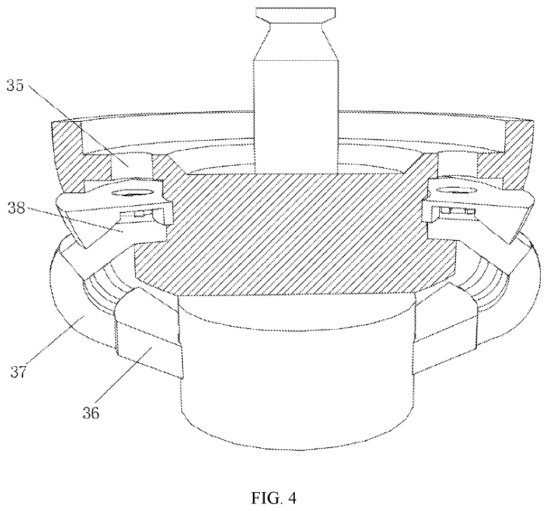

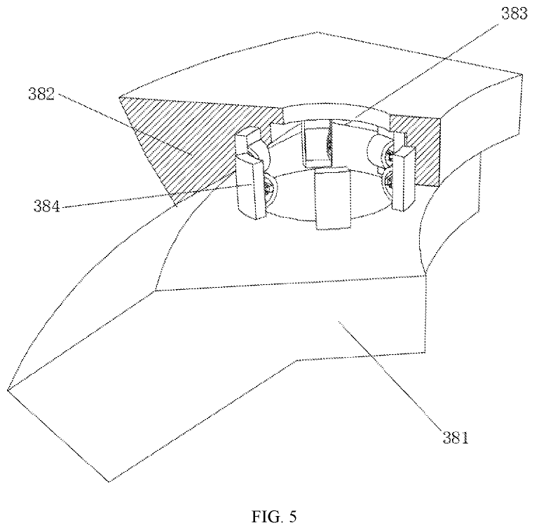

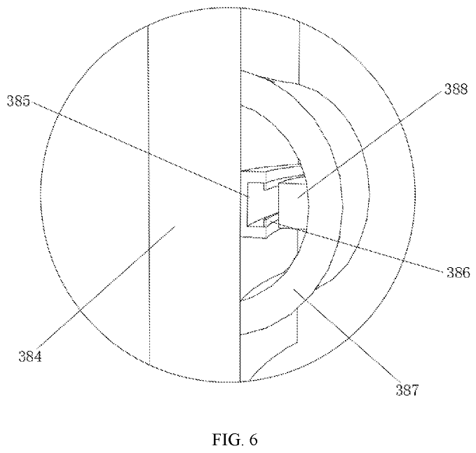

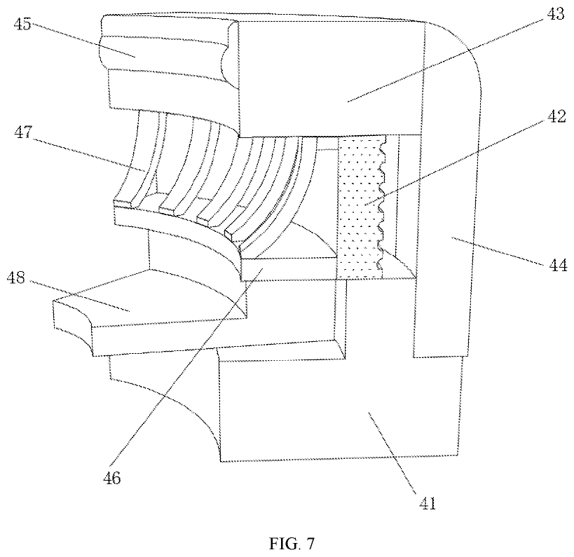

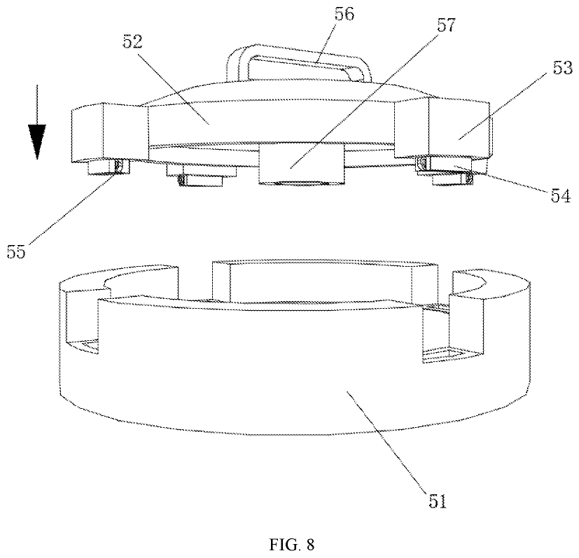

As shown in FIG. 1 through FIG. 8, the present disclosure provides a technical solution: Immobilized enzymolysis equipment for reconstituted rice milk powder includes bottom box 1. A heating device 4 is arranged on an upper surface of the bottom box 1, a splash-proof device 5 is arranged on an upper surface of the heating device 4, and support legs 2 are fixedly connected to corners of a lower surface of the bottom box 1. A rotating motor 6 is arranged on a bottom surface of an inner cavity of the bottom box 1. The rotating motor 6 is provided to achieve the effect of rotating a centrifugal device 3. During the operation of the immobilized enzymolysis equipment, after a test tube is placed on the centrifugal device by an operator, the rotating motor 6 is connected to a power supply and a switch of the rotating motor 6 is turned on, then the rotating motor 6 starts to rotate. The rotating motor 6 penetrates through the bottom surface of the inner cavity of the bottom box 1 and extends to the upper surface of the bottom box 1. The centrifugal device 3 is rotationally connected to one side, extending to the upper surface of the bottom box 1, of the rotating motor 6. The centrifugal device 3 includes a rotating column 31. The rotating column 31 is rotationally connected to an upper surface of the rotating motor 6, and is connected to an output end of the rotating motor 6. A fixed column 32 is fixedly connected to an upper surface of the rotating column 31. A placement plate 33 is fixedly connected to an upper surface of the fixed column 32. A limit column 34 is fixedly connected to an upper surface of the placement plate 33, and an upper placement hole 35 is formed in the upper surface of the placement plate 33. Through the arrangement of the placement plate 33 and the upper placement hole 35, during the operation of the immobilized enzymolysis equipment, a test tube filled with the reconstituted milk powder is placed at the upper placement hole 35 formed in the upper surface of the placement plate 33, such that the test tube can be placed in the centrifugal device 3, and a centrifugal effect can be achieved under the action of the rotating motor 6, so as to achieve the enzymolysis effect on the reconstituted milk powder. A fan-shaped support plate 36 is fixedly connected to an outer surface of the fixed column 32, and a bow-shaped elastic sheet 37 is fixedly connected to one side, away from the fixed column 32, of the fan-shaped support plate 36. The bow-shaped elastic sheet 37 is provided to play a role in buffering during the rotation of the test tube. Vibration may be generated during the rotation of the rotating column 31, and the providing of the bow-shaped elastic sheet 37 may play a certain cushioning effect. A test tube clamping device 38 is arranged at one end, away from the fan-shaped support plate 36, of the bow-shaped elastic sheet 37. The test tube clamping device 38 includes a bottom plate 381 of the clamping device. The bottom plate 381 of the clamping device is fixedly connected to one end, away from the fan-shaped support plate 36, of the bow-shaped elastic sheet 37. A clamping support box 382 is fixedly connected to an upper surface of the bottom plate 381 of the clamping device, and a bottom placement hole 383 is formed in an upper surface of the clamping support box 382. A support plate 384 is provided between an inner cavity of the clamping support box 382 and an opposite face of the bottom plate 381 of the clamping device. A fitting box 385 is fixedly connected to an outer surface of the support plate 384. An adhesive strip 386 is arranged at an inner wall of the fitting box 385. An arc-shaped elastic sheet 387 is fixedly connected to the outer surface of the support plate 384, an extrusion column 388 is fixedly connected to one side, close to the fitting box 385, of the arc-shaped elastic sheet 384, and the extrusion column 388 is in extrusion fit with the fitting box 385. The test tube clamping device 38 is provided to clamp and fix the bottom of the test tube, so as to prevent the test tube from flying off the centrifugal device 3 during rotation. During the operation of the immobilized enzymolysis equipment, the test tube filled with the reconstituted milk powder is placed into the centrifugal device 3 from the upper placement hole 35 formed in the upper surface of the placement plate 33 by the operator, and then the test tube is pressed to be clamped into the bottom placement hole 383 formed in the upper surface of the clamping support box 382. The bottom of the test tube extrudes the arc-shaped elastic sheet 387, and the extrusion column 388 and the fitting box 385 extrude each other during the extrusion. Due to the adhesive strip 386 on the inner wall of the fitting box 385, the adhesive strip 386 and the extrusion column 388 extrude each other, such that the extrusion column 388 is firmly extruded at an inner cavity of the fitting box 385, making the test tube fixed firmly. The heating device 4 includes a base 41 of the heating device. The base 41 of the heating device is fixedly connected to the upper surface of the bottom box 1. A heating motor 42 is arranged on an upper surface of the base 41 of the heating device. A conductive plate 46 is connected to a side surface of the heating motor 42. One end, away from the heating motor 42, of the conductive plate 46 is provided with a heating plate 47, and the heating plate 47 is connected to an output end of the heating motor 42. The heating motor 42 and the heating plate 47 are provided to keep the test tube at a certain temperature. As it is necessary to keep a certain temperature during the growth of enzyme bacteria in the reconstituted rice milk powder, the heating motor 42 and the heating plate 47 are provided to control the temperature around the test tube. During the operation of the immobilized enzymolysis equipment, the heating motor 42 is connected to a power supply by an operator, and then a switch on the heating motor 42 is turned on. The heating motor 42 starts to work, the heating plate 47 dissipates heat to keep the test tube at a certain temperature. A limit top plate 43 is fixedly connected to an upper surface of the heating motor 42, and an anti-collision strip 45 is arranged on an outer surface of the limit top plate 43. A partition plate 44 is fixedly connected to one side, away from the anti-collision strip 45, of the outer surface of the limit top plate 43, and the other end of the partition plate 44 is fixedly connected to a side surface of the base 41 of the heating device. A fan-shaped limit plate 48 is fixedly connected to the upper surface of the base 41 of the heating device. The anti-collision strip 45 is provided to prevent the centrifugal device 3 from colliding with the limit top plate 43 during rotation, because the collision with the limit top plate 43 may lead to nonuniform centrifuging of materials in the test tube, the arrangement of the anti-collision strip 45 can keep the centrifugal device 3 stable when colliding with the limit top plate 43. The fan-shaped limit plate 48 is arranged around the fixed column 32, so as to play a role in limiting the centrifugal device 3. The splash-proof device 5 includes a bottom shell 51 of the splash-proof device. The bottom shell 51 of the splash-proof device is fixedly connected to the outer surface of the limit top plate 43, a top cover 52 of the splash-proof device is sleeved at an inner cavity of the bottom shell 51 the splash-proof device, and a top limit box 57 fixedly connected to a lower surface of the top cover 52 the splash-proof device. The top limit box 57 in frictional fit with the limit column 34. A grip 56 is fixedly connected to an upper surface of the top cover 52 of the splash-proof device. A clamping groove is formed in an upper surface of the bottom shell 51 of the splash-proof device, and a clamping column 53 is fixedly connected to an outer surface of the top cover 52 of the splash-proof device. The clamping column 53 is in extrusion fit with the clamping groove formed in the upper surface of the bottom shell 51 of the splash-proof device. A limit block 54 is fixedly connected to a lower surface of the clamping column 53, and a limit soft sheet 55 is arranged at a side surface of the limit block 54. The limit soft sheet 55 is in extrusion fit with the clamping groove formed in the upper surface of the bottom shell 51 of the splash-proof device. The splash-proof device 5 is provided to prevent liquid in the test tube from splashing everywhere during the rotation of the centrifugal device 3. During the operation of the immobilized enzymolysis equipment, after the test tube is placed on the centrifugal device 3, the top cover 52 of the splash-proof device is placed on the upper surface of the bottom shell 51 of the splash-proof device by the operator. The limit block 54 and the limit soft sheet 55 are placed at the clamping groove formed in the upper surface of the bottom shell 51 of the splash-proof device, and the limit block 54 can be tightly clamped to the clamping groove formed in the upper surface of the bottom shell 51 of the splash-proof device through the limit soft sheet 55, thus achieving a splash-proof effect.

During use, the rotating motor 6 is provided to achieve an effect of rotating the centrifugal device 3. During the operation of the immobilized enzymolysis equipment, after a test tube is placed on the centrifugal device by an operator, the rotating motor 6 is connected to a power supply and a switch of the rotating motor 6 is turned on, then the rotating motor 6 starts to rotate. Through the arrangement of the placement plate 33 and the upper placement hole 35, during the operation of the immobilized enzymolysis equipment, a test tube filled with the reconstituted milk powder is placed at the upper placement hole 35 formed in the upper surface of the placement plate 33, such that the test tube can be placed in the centrifugal device 3, and a centrifugal effect can be achieved under the action of the rotating motor 6, so as to achieve the enzymolysis effect on the reconstituted milk powder. The bow-shaped elastic sheet 37 is provided to play a role in buffering during the rotation of the test tube. Vibration may be generated during the rotation of the rotating column 31, and the providing of the bow-shaped elastic sheet 37 may play a certain cushioning effect The test tube clamping device 38 is provided to clamp and fix the bottom of the test tube, so as to prevent the test tube from flying off the centrifugal device 3 during rotation. During the operation of the immobilized enzymolysis equipment, the test tube filled with the reconstituted milk powder is placed into the centrifugal device 3 from the upper placement hole 35 formed in the upper surface of the placement plate 33 by the operator, and then the test tube is pressed to be clamped into the bottom placement hole 383 formed in the upper surface of the clamping support box 382. The bottom of the test tube extrudes the arc-shaped elastic sheet 387, and the extrusion column 388 and the fitting box 385 extrude each other during the extrusion. Due to the adhesive strip 386 on the inner wall of the fitting box 385, the adhesive strip 386 and the extrusion column 388 extrude each other, such that the extrusion column 388 is firmly extruded at an inner cavity of the fitting box 385, making the test tube fixed firmly. The heating motor 42 and the heating plate 47 are provided to keep the test tube at a certain temperature. As it is necessary to keep a certain temperature during the growth of enzyme bacteria in the reconstituted rice milk powder, the heating motor 42 and the heating plate 47 are provided to control the temperature around the test tube. During the operation of the immobilized enzymolysis equipment, the heating motor 42 is connected to a power supply by an operator, and then a switch on the heating motor 42 is turned on. The heating motor 42 starts to work, the heating plate 47 dissipates heat to keep the test tube at a certain temperature. The anti-collision strip 45 is provided to prevent the centrifugal device 3 from colliding with the limit top plate 43 during rotation, because the collision with the limit top plate 43 may lead to nonuniform centrifuging of materials in the test tube, the arrangement of the anti-collision strip 45 can keep the centrifugal device 3 stable when colliding with the limit top plate 43. The fan-shaped limit plate 48 is arranged around the fixed column 32, so as to play a role in limiting the centrifugal device 3. The splash-proof device 5 is provided to prevent liquid in the test tube from splashing everywhere during the rotation of the centrifugal device 3. During the operation of the immobilized enzymolysis equipment, after the test tube is placed on the centrifugal device 3, the top cover 52 of the splash-proof device is placed on the upper surface of the bottom shell 51 of the splash-proof device by the operator. The limit block 54 and the limit soft sheet 55 are placed at the clamping groove formed in the upper surface of the bottom shell 51 of the splash-proof device, and the limit block 54 can be tightly clamped to the clamping groove formed in the upper surface of the bottom shell 51 of the splash-proof device through the limit soft sheet 55, thus achieving a splash-proof effect.

Apparently, the described embodiments are a part rather than all embodiments. All other embodiments obtained by those of ordinary skill in the art based on the embodiments of the present disclosure without creative efforts shall fall within the protection scope of the present disclosure. The structures, devices and operation methods not specifically described and explained in the present disclosure, unless specifically described and limited, are implemented by conventional means in this field.

What is claimed is:

1. An immobilized enzymolysis equipment for reconstituted rice milk powder, comprising a bottom box, wherein a heating device is arranged on an upper surface of the bottom box, a splash-proof device is arranged on an upper surface of the heating device, support legs are fixedly connected to corners of a lower surface of the bottom box, and a rotating motor is arranged on a bottom surface of an inner cavity of the bottom box; the rotating motor penetrates through the bottom surface of the inner cavity of the bottom box and extends to the upper surface of the bottom box, and a centrifugal device is rotationally connected to one side, extending to the upper surface of the bottom box, of the rotating motor;
the centrifugal device comprises a rotating column, the rotating column is rotationally connected to an upper surface of the rotating motor, the rotating column is connected to an output end of the rotating motor, a fixed column is fixedly connected to an upper surface of the rotating column, a placement plate is fixedly connected to an upper surface of the fixed column, a limit column is fixedly connected to an upper surface of the placement plate, and an upper placement hole is formed in the upper surface of the placement plate.

2. The immobilized enzymolysis equipment for reconstituted rice milk powder according to claim 1, wherein a fan-shaped support plate is fixedly connected to an outer surface of the fixed column, a bow-shaped elastic sheet is fixedly connected to one side, away from the fixed column, of the fan-shaped support plate, and a test tube clamping device is arranged at one end, away from the fan-shaped support plate, of the bow-shaped elastic sheet.

3. The immobilized enzymolysis equipment for reconstituted rice milk powder according to claim 2, wherein the test tube clamping device comprises a bottom plate of the clamping device, which is fixedly connected to one end, away from the fan-shaped support plate, of the bow-shaped elastic sheet, a clamping support box is fixedly connected to the upper surface of the bottom plate of the clamping device, and a bottom placement hole is formed in an upper surface of the clamping support box.

4. The immobilized enzymolysis equipment for reconstituted rice milk powder according to claim 3, wherein a support plate is arranged between the inner cavity of the clamping support box and an opposite face of the bottom plate of the clamping device, a fitting box is fixedly connected to an outer surface of the support plate, an adhesive strip is arranged at an inner wall of the fitting box, an arc-shaped elastic sheet is fixedly connected to the outer surface of the support plate, an extrusion column is fixedly connected to one side, close to the fitting box, of the arc-shaped elastic sheet, and the extrusion column is in extrusion fit with the fitting box.

5. The immobilized enzymolysis equipment for reconstituted rice milk powder according to claim 1, wherein the heating device comprises a base of the heating device, the base of the heating device is fixedly connected to the upper surface of the bottom box; a heating motor is arranged on an upper surface of the base of the heating device; a conductive plate is connected to a side surface of the heating motor; one end, away from the heating motor, of the conductive plate is provided with a heating plate; and the heating plate is connected to an output end of the heating motor.

6. The immobilized enzymolysis equipment for reconstituted rice milk powder according to claim 5, wherein a limit top plate is fixedly connected to an upper surface of the heating motor, an anti-collision strip is arranged on an outer surface of the limit top plate, a partition plate is fixedly connected to one side, away from the anti-collision strip, of the outer surface of the limit top plate, the other end of the partition plate is fixedly connected to a side surface of the base of the heating device, and a fan-shaped limit plate is fixedly connected to the upper surface of the base of the heating device.

7. The immobilized enzymolysis equipment for reconstituted rice milk powder according to claim 1, wherein the splash-proof device comprises a bottom shell of the splash-proof device; the bottom shell of the splash-proof device is fixedly connected to the outer surface of the limit top plate, a top cover of the splash-proof device is sleeved at an inner cavity of the bottom shell of the splash-proof device, a top limit box is fixedly connected to a lower surface of the top cover of the splash-proof device, and the top limit box is in frictional fit with the limit column.

8. The immobilized enzymolysis equipment for reconstituted rice milk powder according to claim 7, wherein a grip is fixedly connected to an upper surface of the top cover of the splash-proof device, a clamping groove is formed in an upper surface of the bottom shell of the splash-proof device, a clamping column is fixedly connected to an outer surface of the top cover of the splash-proof device, the clamping column is in extrusion fit with the clamping groove formed in the upper surface of the bottom shell of the splash-proof device, a limit block is fixedly connected to a lower surface of the clamping column, and a limit soft sheet is arranged at a side surface of the limit block, and the limit soft sheet is in extrusion fit with the clamping groove formed in the upper surface of the bottom shell of the splash-proof device.
